(19) United States
(12) Patent Application Publication
(10) Pub. No.: US 2018/0127328 A1
(43) Pub. Date: May 10, 2018

(54) THERMAL PRE-IGNITION AGENT

(71) Applicant: RUAG Ammotec GmbH, Fürth (DE)

(72) Inventors: Aleksej HOSCHENKO, Fürth (DE); Ulrich BLEY, Fürth (DE); Peter Simon LECHNER, Oberasbach (DE)

(21) Appl. No.: 15/525,757

(22) PCT Filed: Nov. 10, 2015
(86) PCT No.: PCT/EP2015/076245

§ 371 Date: Dec. 1, 2017

(30) Foreign Application Priority Data
Nov. 10, 2014 (DE) 10 2014 016 513.2

(51) Int. Cl. C06B 25/04; C06C 7/00; C06C 9/00

(52) U.S. Cl. CPC C06B 25/04; C07D 273/02; C06C 9/00; C06C 7/00

(57) ABSTRACT

The invention relates to a thermal pre-ignition agent which contains as components 20 to 50 wt. % of dinitrobenzofuroxane and 50 to 80 wt. % of an oxidizing agent and a nitrogen-containing compound.

The present invention relates to a thermal pre-ignition agent.

Thermal pre-ignition agents in the sense of the invention are pyrotechnic substances or substance mixtures that have ignition temperatures below 200° C.

Fields of application of thermal pre-ignition agents of this kind include, for example, safety systems, preferably thermal fuses in gas generators or separator elements for batteries. Safety systems of this kind are in turn preferably used in motor vehicles.

The thermal pre-ignition agents are used in order to ignite, in a controlled manner, the mixtures of a gas generator in the event of a vehicle fire, which mixtures generally generate gas that is thermally very stable.

Thermal pre-ignition agents are also used as pyrotechnic matter in separator elements, preferably for battery clamps. These separator elements are intended to interrupt the power supply in the event of a fire, in particular a vehicle fire, or in the event of a motor vehicle accident, in which the gas generator is triggered.

The disadvantage of thermal pre-ignition agents containing nitrocellulose and of propellant powder derived therefrom lies in the fact that these mixtures have ignition temperatures of approximately 160° C. and have insufficient long-term stability. Mixtures based on nitrotriazolone and guanidine nitrate, which are free from nitrocellulose, are described in document DE 197 30 873 A1. Pre-ignition powders which demonstrate a weight loss<₃ weight-% even after warm storage over 400 hours at 107° C. and which have a self-ignition temperature (decomposition temperature) in the range between 150° C. and 200° C. are also known from document WO 99/41213.

The object of the present invention is to provide an alternative thermal pre-ignition agent having an explosion temperature<180° C., a friction sensitivity>250 N, an impact sensitivity>3 J, and a long-term stability expressed as weight loss<1 weight-% after warm storage at 125° C. over 1000 hours. The terms ignition temperature, self-ignition temperature, decomposition temperature and explosion temperature are used synonymously in the sense of the present invention.

A further object of the present invention was to provide a thermal pre-ignition agent which can be used for gas generators in motor vehicle safety systems or in separator elements for battery clamps.

This object is surprisingly achieved in accordance with the invention by the features of the main claim. Preferred embodiments can be found in the dependent claims.

It has surprisingly been found that with mixtures of from 20 to 50 weight-% of dinitrobenzofuroxane (DNBF) with 50 to 80 weight-% of an oxidizing agent and a compound comprising nitrogen, the explosion temperatures can be controlled in the range of 140° C. to 175° C. The explosion temperatures of the mixtures can be lower than those of the individual components.

The melting point or decomposition point of pure DNBF is for example approximately 170° C.

The oxidizing agent and compounds comprising nitrogen are selected from the groups consisting of:
1. Oxidizing agent (individually or in mixtures)
   nitrates of alkali metals or of alkaline earth metals or of ammonium, such as sodium, potassium or ammonium nitrate, perchlorates of alkali metals or of alkaline earth metals or of ammonium, peroxides of alkali metals or of alkaline earth metals or of zinc.
2. Compounds comprising nitrogen (individually or in mixtures)
   ammonium picrate, aminoguanidinium picrate, guanidinium picrate, aminoguanidinium styphnate, guanidinium styphnate, nitroguanidine, nitroaminoguanidine, nitrotriazolone, derivates of tetrazole such as 5-aminotetrazole, ditetrazolylamine, ditetrazole and the salts thereof, nitraminotetrazole and the salts thereof such as ammonium nitraminotetrazol ate and aminoguanidinium-nitraminotetrazolate, aminoguanidine nitrate, diaminoguanidine nitrate, triaminoguanidine nitrate, guanidine nitrate, dicyandiamidine nitrate, diaminoguanidine azotetrazolate, triaminoguanidine azotetrazolate or ammonium azotetrazolate.

Further additives can be contained, selected from the groups consisting of:
3. Reducing agent (individually or in mixtures)
   aluminum, titanium, titanium hydride, boron, boron hydride, zirconium, zirconium hydride, silicon, graphite, active carbon, carbon black.
4. Binding agent (individually or in mixtures)
   cellulose and the derivates thereof, polyvinyl butyrals, polynitropolyphenylene, polynitrophenyl ether, plexigum, polyvinyl acetate, copolymers.
5. Burning rate modifiers, stabilizers and processing aids (individually or in mixtures)
   ferrocene and derivates, acetonyl acetates, salicylates, barium carbonate, strontium carbonate, magnesium carbonate, melamine, zinc oxide, zinc carbonate, silicates, silica gels, silica acids, boron nitride.

The production and processing are performed in accordance with standard methods known per se. These methods include, for example, tempering, extrusion, pelleting or granulation.

More specifically, the present invention relates to:
a thermal pre-ignition agent which comprises, as components, 20 to 50 weight-% dinitrobenzofuroxane and 50 to 80% weight-% of an oxidizing agent and a compound comprising nitrogen;
a thermal pre-ignition agent which comprises 30 to 70 weight-% of an oxidizing agent selected from one or more of the list comprising nitrates of alkali metals and/or of alkaline earth metals and/or of ammonium, perchlorates of alkali metals and/or of alkaline earth metals and/or of ammonium, peroxides of alkali metals and/or of alkaline earth metals and/or of zinc;
a thermal pre-ignition agent which comprises 10 to 50 weight-% of a nitrogen-containing compound, selected from one or more of the list comprising ammonium picrate, aminoguanidinium picrate, guanidinium picrate, aminoguanidinium styphnate, guanidinium styphnate, nitroguanidine, nitroaminoguanidine, nitrotriazolone, derivates of tetrazole and/or the salts thereof, nitraminotetrazole and/or the salts thereof, aminoguanidine nitrate, diaminoguanidine nitrate, triaminoguanidine nitrate, guanidine nitrate, dicyandiamidine nitrate, diaminoguanidine azotetrazolate;
a thermal pre-ignition agent which comprises 1 to 15 weight-% of a reducing agent selected from one or more of the list comprising aluminum, titanium, titanium hydride, boron, boron hydride, zirconium, zirconium hydride, silicon, graphite, active carbon, carbon black;
a thermal pre-ignition agent which comprises 1 to 20 weight-% of a binding agent selected from one or more of the list comprising cellulose and the derivates thereof, polyvinyl butyrals, polynitropolyphenylene, polynitrophenyl ether, plexigum, polyvinyl acetate and the copolymers thereof;
a thermal pre-ignition agent which comprises 0.1 to 15 weight-%, particularly preferably 1 to 10 weight-%, burning rate modifiers and processing aids selected from one or more of the list comprising ferrocene and its derivates, acetonyl acetates, salicylates, barium carbonate, strontium carbonate, magnesium carbonate, melamine, zinc oxide, zinc carbonate, silicates, silica gels, silicic acids and/or boron nitride;
use of the thermal pre-ignition agent according to the invention as a thermal fuse;
use of the thermal pre-ignition agent according to the invention in motor vehicle safety systems;
use of the thermal pre-ignition agent according to the invention in gas generators;
use of the thermal pre-ignition agent according to the invention in separator elements, preferably for battery clamps.

The invention will be explained in greater detail by the following examples, without being limited thereto:

Table 1 shows the compositions of 6 different mixtures of the thermal pre-ignition agent. The components were weighed in the specified weight ratios (values in weight percent (weight-%)) into plastic containers and homogenized for 30 minutes in a tumble mixer.

TABLE 1
Mixtures

| | | Guanidine | Sodium | Potassium | |
|---|---|---|---|---|---|
| | DNBF | nitrate | nitrate | nitrate | Other |
| | [weight- | [weight- | [weight- | [weight- | [weight- |
| Mixture | %] | %] | %] | %] | %] |
| 1 | 30 | 20 | 50 | | |
| 2 | 30 | 20 | | 50 | |
| 3 | 30 | | 50 | | 20^{a} |
| 4 | 30 | | 50 | | 20^{b} |
| 5 | 30 | | 50 | | 20^{c} |
| 6 | 30 | | 50 | | 20^{d} |

^{a}triaminoguanidine nitrate
^{b}aminoguanidine nitrate
^{c}nitroguanidine
^{d}dicyandiamidine dinitrate

Table 2 shows the explosion points, friction and impact sensitivities of the mixtures. The friction and impact sensitivities were measured by methods as stipulated by the Bundesanstalt für Materialforschung (BAM) (German Federal Institute for Materials Research), whereas the explosion points were measured by thermogravimetric analysis (Mettler) at a heating rate of 10° C. per minute.

TABLE 2
Overview of explosion temperatures,
friction and impact sensitivities

| | Explosion | Friction | Impact |
|---|---|---|---|
| | temperature | sensitivity | sensitivity |
| Mixture | [° C.] | [N] | [J] |
| 1 | 158 | >360 | 5 |
| 2 | 167 | >360 | 5 |
| 3 | 145 | >360 | 5 |
| 4 | 150 | >360 | 5 |
| 5 | 172 | >360 | 5 |
| 6 | 170 | >360 | 5 |

Table 3 shows the weight losses and explosion points after thermal load (24 h, 125° C. and 400 h, 110° C.) of some mixtures selected from the examples. The weight loss was measured similarly to the Holland test. The explosion points were measured by thermogravimetric analysis (Mettler) at a heating rate of 10° C. per minute.

Low weight losses of from 0.1 to 0.7 weight-% were observed after 400 h, with no significant change to the explosion temperature after thermal load.

TABLE 3
Overview of weight losses and explosion temperatures

| | | Explosion temperature |
|---|---|---|
| | Weight loss | after load |
| Storage | [weight-%] | [° C.] |
| 5000 h/100° C. | <0.1 | 159 |
| 1000 h/110° C. | <0.1 | 159 |
| 500 h/120° C. | <0.2 | 157 |
| 1000 h/125° C. | <0.5 | 155 |

The results show that the pyrotechnic agents defined in accordance with the invention have explosion temperatures in the range of from 172 to 191° C. and are considered to be stable in accordance with the requirements of the automotive industry.

1. A thermal pre-ignition agent, comprising 20 to 50 weight-% dinitrobenzofuroxane and 50 to 80% weight-% of an oxidizing agent and a compound comprising nitrogen.

2. The thermal pre-ignition agent according to claim 1, wherein it comprises 30 to 70 weight-% of an oxidizing agent selected from one or more of the list comprising nitrates of alkali metals and/or of alkaline earth metals and/or of ammonium, perchlorates of alkali metals and/or of alkaline earth metals and/or of ammonium, peroxides of alkali metals and/or of alkaline earth metals and/or of zinc.

3. The thermal pre-ignition agent according to claim 1, wherein it comprises 10 to 50 weight-% of a nitrogen-containing compound, selected from one or more of the list comprising ammonium picrate, aminoguanidinium picrate, guanidinium picrate, aminoguanidinium styphnate, guanidinium styphnate, nitroguanidine, nitro amino guanidine, nitrotriazolone, derivates of tetrazol and/or the salts thereof, nitraminotetrazole and/or the salts thereof, aminoguanidine nitrate, di-aminoguanidine nitrate, tri-aminoguanidin nitrate, guanidine nitrate, dy-cyanidiamindine nitrate, di-aminoguanidine-azotetrazolate.

4. The thermal pre-ignition agent according to claim 1, wherein it comprises 1 to 15 weight-% of a reducing agent selected from one or more of the list comprising aluminum, titanium, titanium hydride, boron, boron hydride, zirconium, zirconium hydride, silicon, graphite, active carbon, carbon black.

5. The thermal pre-ignition agent according to claim 1, wherein it comprises 1 to 20 weight-% of a binding agent selected from one or more of the list comprising cellulose and the derivates thereof, polyvinyl butyrals, polynitropolyphenylene, polynitrophenyl ether, plexigum, polyvinyl acetate and the copolymers thereof.

6. The thermal pre-ignition agent according to claim 1, wherein it comprises 0.1 to 15 weight-%, particularly preferred 1 to 10 weight-%, burning rate modifiers and processing aids selected from one or more of the list comprising ferrocene and its derivates, acetonyl acetates, salicylates, barium carbonates, strontium carbonates, magnesium carbonates, melamine, zinc oxide, zinc carbonate, silicates, silica gels, and/or borium nitrid.

7. A thermal fuse comprising the thermal pre-ignition agent according claim 1.

8. A motor vehicles safety system comprising the thermal pre-ignition agent according claim lone or more of the claim 1.

9. A gas generator comprising the thermal pre-ignition agent according claim 1.

10. A separator element comprising the thermal pre-ignition agent according claim 1.

11. A separator element for a battery clamp comprising the thermal pre-ignition agent according claim 1.
